(19) United States
(12) Patent Application Publication
(10) Pub. No.: US 2023/0287479 A1
(43) Pub. Date: Sep. 14, 2023

(54) IDENTIFYING TARGET NUCLEIC ACIDS USING IMMOBILIZED NUCLEASE

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US)

(73) Assignee: Hewlett-Packard Development Company, L.P., Spring, TX (US)

(21) Appl. No.: 18/016,860

(22) PCT Filed: Jul. 21, 2020
(86) PCT No.: PCT/US2020/042944

§ 371 Date: Jan. 18, 2023

(51) Int. Cl. C12Q 1/6823; B01L 3/00; B01L 7/00

(52) U.S. Cl. CPC C12Q 1/6823; B01L 3/502761; B01L 7/00; B01L 2200/0652; B01L 2200/16; B01L 2300/0654; B01L 2400/0487; B01L 2300/088; B01L 2300/1805

(57) ABSTRACT

An example method, consistent with the present disclosure, includes receiving in a microfluidic channel, a fluid sample and a reagent, where the reagent includes a reporter nucleic acid labeled with a detectable ligand. The method further includes identifying a target nucleic acid in the fluid sample using a guide ribonucleic acid (gRNA) and a programmable nuclease immobilized in a side channel fluidically coupled to the microfluidic channel. Responsive to identifying the target nucleic acid in the fluid sample, the method includes causing cleavage of the detectable ligand, and detecting the detectable ligand from the cleaved reporter nucleic acid in the side channel.

### BACKGROUND

Analytical probes and detection optics can be used in detection of particular substrates from samples. The physical process of analyzing the samples and particular method used can impact the information learned about a particular sample being analyzed. As nucleic acid diagnostics become increasingly relevant for a variety of healthcare applications, detection technologies that enable multiplexing with a high specificity and sensitivity at low cost would be of great utility in both clinical and research settings.

### BRIEF DESCRIPTION OF THE DRAWINGS

FIG. 1 illustrates a flow chart of an example method for identifying target nucleic acids using immobilized nuclease, consistent with the present disclosure.

FIG. 2A illustrates an example apparatus for identifying target nucleic acids using immobilized nuclease, consistent with the present disclosure.

FIG. 2B illustrates an example apparatus for identifying target nucleic acids using immobilized nuclease, consistent with the present disclosure.

FIG. 3 illustrates an example apparatus for identifying target nucleic acids using immobilized nuclease, including a sample reservoir and a reagent reservoir, consistent with the present disclosure.

FIG. 4 illustrates an example apparatus for identifying target nucleic acids using immobilized nuclease, including a sample reservoir and a reagent reservoir, and a collector, consistent with the present disclosure.

FIG. 5A illustrates an example apparatus for identifying target nucleic acids, including a looped side channels, consistent with the present disclosure.

FIG. 5B illustrates an example apparatus for identifying target nucleic acids, including a looped side channels, consistent with the present disclosure.

FIG. 6 illustrates an example apparatus for identifying target nucleic acids, including looped side channels and additional pumps, consistent with the present disclosure.

FIG. 7 illustrates an example apparatus for identifying target nucleic acids, including looped side channels and additional pumps, consistent with the present disclosure.

FIG. 8 illustrates an example apparatus for identifying target nucleic acids, including looped side channels and electrochemical reporters, consistent with the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Rapid, accurate, cost-effective detection and identification of pathogens is important for the control and management of infectious disease. However, diagnostics that implement processing of samples in a centralized laboratory slow diagnosis in resource-limited settings. Therefore, the development of reliable and rapid diagnostic tests that can be conducted outside the clinical laboratory may improve disease treatment and management. Development of rapid initial screening tests that can be used at the point-of-care may minimize the delay and expense of testing in centralized laboratories.

Nucleic acid analysis, specifically detection of specific sequences, forms the backbone of many biochemical assays, including those for pathogen detection and other disease diagnostics. One method of nucleic acid analysis is polymerase chain reaction (PCR). PCR is both highly sensitive and highly specific, but often requires tight thermal control of the amplification reaction, design of multiple primers, and often removal of PCR inhibitors from the sample to be amplified. This thermal control results in slow amplification processes by PCR and other amplification processes. Moreover, PCR and other (isothermal) nucleic acid amplification and detection methods have a high limit of detection (LoD) (up to single nucleic acid molecule) and are often limited to detecting 4-5 target nucleic acid sequences in a same testing volume.

Current diagnostic methods rely heavily on culturing, biomarker analysis via PCR and antibody-based methods, and even genome sequencing. All nucleic acid amplification methods use expensive reagents, complicated tools, require trained personnel, are time consuming and usually labor intensive. Identifying target nucleic acids using immobilized nuclease, consistent with the present disclosure, can benefit from fluidic automation and multiplexing to reduce the time and labor commitment associated with nucleic acid detection. Identifying target nucleic acids using immobilized nuclease, provides a multiplexed method and apparatus in which pre-analysis sample preparation is reduced or eliminated, and the use of multiple primers and reporters may be reduced or eliminated. Moreover, identifying target nucleic acids using immobilized nuclease, consistent with the present disclosure, allows for potentially thousands of target nucleic acid sequences to be analyzed in a same testing volume and at a same time, as opposed to being limited to detecting 4-5 target nucleic acid sequences with the aforementioned methodologies.

Currently, there are three different editing nucleases available: Zinc Finger Nucleases (ZFNs), transcription activator-like effector nucleases (TALENs), and clustered regularly interspaced short palindromic repeats (CRISPR). ZFNs are restriction enzymes generated by fusing a zinc finger deoxyribonucleic acid (DNA)-binding domain to a DNA-cleavage domain. Zinc finger domains can be engineered to target specific desired DNA sequences and this enables ZFNs to target unique sequences within complex genomes. TALENs are restriction enzymes that can be engineered to cut specific sequences of DNA. They are made by fusing a TAL effector DNA-binding domain to a DNA cleavage domain. Transcription activator-like effectors (TALEs) can be engineered to bind to practically any desired DNA sequence, so when combined with a nuclease, DNA can be cut at specific locations. CRISPR is a family of DNA sequences found in the genomes of prokaryotic organisms such as bacteria and archaea. The Cas (or “CRISPR-associated nuclease”) is an enzyme that uses CRISPR sequences as a guide to recognize and cleave specific strands of DNA that are complementary to the CRISPR sequence.

CRISPR recognizes a target nucleic acid through a short RNA sequence known as guide RNA (gRNA) and double-strand breaks are produced by a Cas protein. A variety of different Cas proteins may be used. The CRISPR/Cas system is classified into two main classes that are further subdivided into different types and subtypes based on the organization of their loci and signature proteins. Class I CRISPR/Cas systems include type I, III, and IV, which employ multisubunit effector complexes. Class II CRISPR/Cas systems use single, RNA-guided, multidomain Cas proteins to recognize and cleave target sequences. Class II CRISPR/Cas systems encompass multiple types, including type II systems such as Cas9, type V, including subtypes Cas12 and Cas14 (designated now as Cas12f), and type VI, including Cas13 systems.

After recognition of a target sequence, guided by a gRNA, a Cas protein cleaves the target DNA, creating a site-specific DNA doublestrand break (DSB). The structure of the gRNA scaffold depends on the Cas protein used. The CRISPR/Cas9 system is the most widely used for genome engineering applications. However, different Cas enzymes have different activities that can be advantageous for diagnostic applications. CRISPR/Cas12a produces staggered-end DSBs, CRISPR/Cas13 targets single-stranded RNA (ssRNA), and CRISPR/Cas14 targets single-stranded DNA (ssDNA), to name a few.

Consistent with the present disclosure, each of ZFNs, TALENs, and CRISPR/Cas may be used for identification of nucleic acids. Particularly, nucleases for the particular application may be immobilized on a substrate and, in combination with various imaging techniques, may be used to identify the presence of various nucleic acids of interest.

Identifying target nucleic acids using immobilized nuclease, consistent with the present disclosure, can benefit from fluidic automation and multiplexing. Consistent with the present disclosure, specific detection of nucleic acids may be performed via target-activated collateral cleavage of nucleic acids using programmable nucleases.

An example method, consistent with the present disclosure, includes receiving in a microfluidic channel, a fluid sample and a reagent, where the reagent includes a reporter nucleic acid labeled with a detectable ligand. The method further includes identifying a target nucleic acid in the fluid sample using a guide ribonucleic acid (gRNA) and a programmable nuclease immobilized in a side channel fluidically coupled to the microfluidic channel. The method includes responsive to identifying the target nucleic acid in the fluid sample, causing cleavage of the detectable ligand, and detecting the detectable ligand from the cleaved reporter nucleic acid in the side channel.

An example apparatus for identifying target nucleic acids using immobilized nuclease, consistent with the present disclosure, includes a sample input to receive a fluid sample, and a reagent input to receive a reagent for nucleic acid testing, where the reagent includes a reporter nucleic acid labeled with a detectable ligand. The apparatus further includes a microfluidic channel fluidically coupled to the sample input and the reagent input, and a plurality of side channels fluidically coupled to the microfluidic channel, wherein each side channel includes a programmable nuclease and a gRNA immobilized in the respective side channel.

Another example apparatus for identifying target nucleic acids using immobilized nuclease, consistent with the present disclosure, includes a microfluidic channel including a sample input to receive a fluid sample and a reagent input to receive a reagent for nucleic acid testing, where the reagent includes a reporter nucleic acid labeled with a detectable ligand. The apparatus includes a plurality of side channels, each side channel forming a loop fluidically coupled to the microfluidic chamber. Each loop includes a pump, and a programmable nuclease and a gRNA immobilized in the respective side channel.

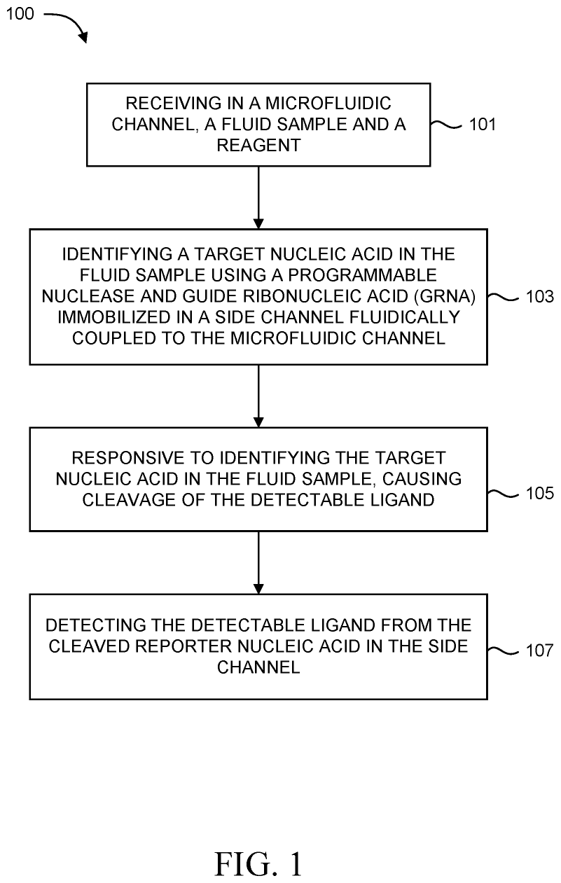

Turning now to the figures, FIG. 1 illustrates a flow chart of an example method 100 for identifying target nucleic acids using immobilized nuclease, consistent with the present disclosure. The method 100 includes at 101 receiving in a microfluidic channel, a fluid sample and a reagent, wherein the reagent includes a reporter nucleic acid labeled with a detectable ligand. At 103, the method 100 includes identifying a target nucleic acid in the fluid sample using a gRNA and a programmable nuclease immobilized in a side channel fluidically coupled to the microfluidic channel. At 105, the method 100 includes causing cleavage of the detectable ligand responsive to identifying the target nucleic acid in the fluid sample. At 107, the method 100 includes detecting the detectable ligand from the cleaved reporter nucleic acid in the side channel.

As used herein, a programmable nuclease refers to or includes an enzyme designed to recognize and cleave a particular nucleic acid sequence. Based on the type of programmable nuclease used, different methodologies may be used to detect the cleavage activity of the nuclease. For example, following recognition and cleavage of the specific target, Cas12a, Cas13, and Cas14 exhibit collateral, nonspecific activities against single stranded DNA (ssDNA) or single stranded RNA (ssRNA). The binding of the programmable nuclease to its target DNA results in nonspecific ssDNA (or ssRNA as the case may be) cleavage activity, which completely degrades other existing ssDNA molecules.

By identifying a nucleic acid in a sample using imbedded nucleases, as discussed herein, signal amplification identifying presence of the target nucleic acid may be decoupled from the target amplification, allowing for greater signal amplification, and a limit of detection in the attomolar range may be achieved.

In some examples, a plurality of side channels are fluidically coupled to the microfluidic channel, each side channel including a respective pump. In such examples, the method includes moving the fluid sample into each of the plurality of side channels by actuating the pumps in the side channels. The method 100 may further include moving the fluid sample into each of the plurality of side channels in a sequential order by independently actuating the respective pumps in the sequential order.

Various additional pre-or-post assay steps may be performed, consistent with the present disclosure. For instance, in some examples, the method 100 includes filtering, using plurality of filtration structures disposed orthogonal to the flow of the fluid sample in the microfluidic channel, polymerase or nuclease inhibiting compounds. Moreover, various steps within the process described may be performed in a variety of ways. For instance, in some examples the detectable ligand is a fluorophore, and the reporter nucleic acid is labeled with the fluorophore and a quencher. Additionally and/or alternatively, in some examples the detectable ligand is an electrochemical reporter probe. Based on which detectable ligand is used, a different respective imaging method may be used, as discussed further herein.

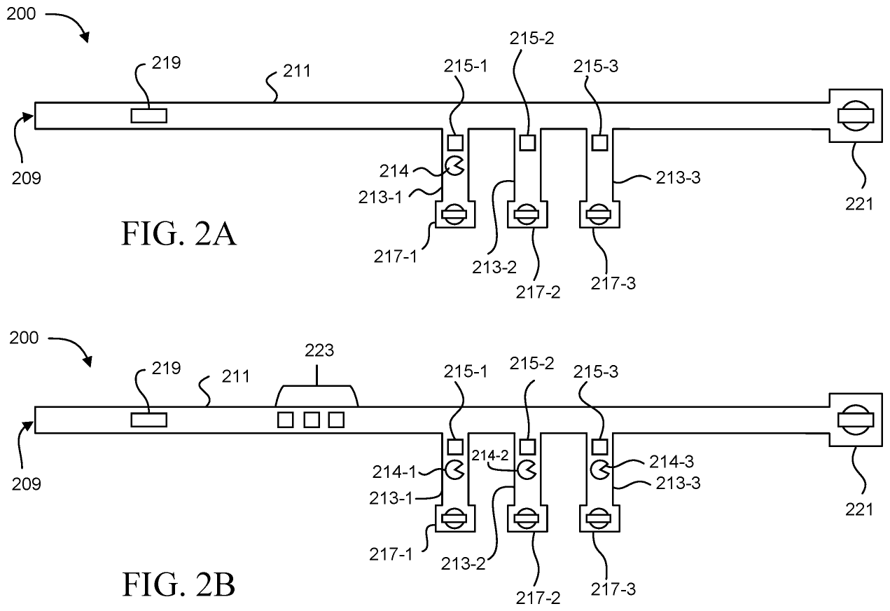

FIG. 2A illustrates an example apparatus 200 for identifying target nucleic acids using immobilized nuclease, consistent with the present disclosure. The apparatus 200 is capable of performing the method 100 illustrated in FIG. 1. For instance, as illustrated in FIG. 2A, the apparatus 200 includes a sample input 209 to receive a fluid sample. A fluid sample, as used herein, generally refers to or includes any material containing nucleic acid, including for example foods and allied products, clinical, and environmental samples. However, the fluid sample will generally be a biological sample, which may contain any viral or cellular material, including all prokaryotic or eukaryotic cells, viruses, bacteriophages, mycoplasmas, protoplasts, and organelles. Such biological material may thus comprise all types of mammalian and non-mammalian animal cells, plant cells, algae including blue-green algae, fungi, bacteria, protozoa, etc. Representative samples thus include whole blood and blood-derived products such as plasma, serum and buffy coat, urine, feces, cerebrospinal fluid or any other body fluids, tissues, cell cultures, cell suspensions, etc. The fluid sample may comprise a lysate. The fluid sample may also include relatively pure starting material such as a PCR product or semi-pure preparations obtained by other nucleic acid recovery processes.

The apparatus 200 further includes a reagent input to receive a reagent for nucleic acid testing. In the example illustrated in FIG. 2A, the reagent input is also the sample input (e.g., 209), although examples are not so limited. As discussed further herein, different locations may be provided for receiving the fluid sample and for receiving the reagent or reagents. These reagents may include short nucleic acid sequences, such as 5 base pairs, with a quencher and a fluorophore. The reagents may also include non-specific primers, polymerase, deoxyribonucleotide triphosphates (dNTPs), and/or a buffer for a nucleic acid amplification reaction such as PCR prior to the nuclease identification reaction. A variety of different reagents may be used, depending on the analysis to be performed. For instance, in some examples the reagent includes a reporter nucleic acid labeled with a detectable ligand. As such, the apparatus 200 may include a reagent input to receive a reagent for nucleic acid testing, wherein the reagent includes a reporter nucleic acid labeled with a detectable ligand.

As discussed above, several components and reagents may be used in PCR or other amplification reactions, and therefore may be included in the reagents input to apparatus 200. Among these components are, a nucleic acid template, such as a DNA template (e.g., double-stranded DNA) that contains the target sequence to be amplified, an enzyme that polymerizes new nucleic acid strands (e.g., a polymerase enzyme such as DNA polymerase, e.g., Taq DNA polymerase), two nucleic acid primers (oligonucleotides, e.g., single-stranded) that are complementary to the 3′ (three prime) ends of each of the sense and antisense strands of the nucleic acid target, nucleoside triphosphates (NTPs) such as dNTPs and ribonucleoside triphosphates (rNTPs), and a buffer solution providing a suitable chemical environment for amplification and optimum activity and stability of the polymerase. Specific buffer solutions may include bivalent cations, such as magnesium (Mg) or manganese (Mn) ions, and monovalent cations such as potassium (K) ions.

The apparatus 200 further includes a microfluidic channel 211 fluidically coupled to the sample input 209 and the reagent input 209. The microfluidic channel 211 may be formed by performing etching, microfabrication processes (e.g., photolithography), or micromachining processes in a substrate. Accordingly, microfluidic channel 211 may be defined by surfaces fabricated in the substrate of apparatus 200.

In some examples, the microfluidic channel 211 may be of sufficiently small size (e.g., of nanometer sized scale, micrometer sized scale, millimeter sized scale, etc.) to facilitate pumping of small volumes of fluid (e.g., picoliter scale, nanoliter scale, microliter scale, milliliter scale, etc.). For example, the microfluidic channel 211 may facilitate capillary pumping due to capillary force.

The apparatus 200 may further include a plurality of side channels 213-1, 213-2, and 213-3 (referred to collectively as side channels 213) fluidically coupled to the microfluidic channel 211. Similar to microfluidic channel 211, side channels 213 may be formed by performing etching, microfabrication processes (e.g., photolithography), or micromachining processes in a substrate. Also, side channels 213 may, be of sufficiently small size (e.g., of nanometer sized scale, micrometer sized scale, millimeter sized scale, etc.) to facilitate pumping of small volumes of fluid (e.g., picoliter scale, nanoliter scale, microliter scale, milliliter scale, etc.).

In various examples, each side channel includes a nuclease 214 and a gRNA in the respective side channel, where the nuclease 214 is immobilized in the side channel. A particular gRNA, selected for the particular nuclease used, may be provided either with the reagents at input 209, or into each side channel. For instance, side channel 213-1 may include a first nuclease immobilized therein and may receive a first gRNA, side channel 213-2 may include a second nuclease immobilized therein and may receive a second gRNA, and side channel 213-3 may include a third nuclease immobilized therein and may receive a third gRNA. As such, the apparatus 200 may include a plurality of side channels fluidically coupled to the microfluidic channel, wherein each side channel includes a gRNA and a programmable nuclease immobilized in the respective side channel.

As different nucleases may cleave nucleic acid sequences at different temperatures, in various examples, each respective side channel includes a respective heater disposed proximal to the immobilized nuclease. For instance, side channel 213-1 may include a first heater 215-1, side channel 213-2 may include a second heater 215-2, and side channel 213-3 may include a third heater 215-3 (collectively referred to herein as heaters 215). Each of the plurality of heaters 215 may be independently controlled, such that each respective heater may be set to a different respective temperature.

Additionally, each of the side channels 213 may include a guide RNA (gRNA) for a particular target that is injected via wax removal ports (not illustrated) into the appropriate channel. For instance, a wax removal port on side channel 213-1 may receive a first gRNA, a wax removal port on side channel 213-2 may receive a second gRNA, and a wax removal port on side channel 213-3 may receive a third gRNA.

Each of the side channels 213 may include a nozzle for ejecting sample therefrom, and for moving sample through the respective side channel. For instance, side channel 213-1 may include a first nozzle 217-1, side channel 213-2 may include a second nozzle 217-2, and side channel 213-3 may include a third nozzle 217-3 (collectively referred to herein as nozzles 217). For instance, an integrated pull pump such as a thermal inkjet (TIJ) nozzle may draw sample fluid from a sample reservoir, and reagents from a reagent reservoir. In such a manner, gRNA can be loaded in the respective side channel by loading the reagent reservoir with a particular gRNA and firing the appropriate dispenser pump to draw the particular gRNA into appropriate chamber. As an illustration, side channel 213-1 may include nuclease 214 immobilized therein, and may be used for detecting a particular nucleic acid sequence in a fluid sample. The fluid sample may be input at 209, as well as reagents. Among the reagents input at 209 may include a gRNA specific for the nuclease 214, and which allows the target nucleic acid sequence to be identified using the nuclease 214 and the gRNA. To draw the gRNA and fluid sample into side channel 213-1, nozzle 217-1 may fire. A different respective nuclease may be immobilized in side channel 213-2 and 213-3, respectively, and nozzles 217-2 and 217-3 may fire to draw sample and reagent into the associated side channel.

As illustrated in FIG. 2A, the apparatus 200 may also include a pump 219 to move the fluid sample through the apparatus 200, as well as a pump 221 to eject the fluid sample. Pump 219 may include a piezoelectric inkjet (PIJ) resistor, a TIJ resistor, or other type of embedded pump. Similarly, although pump 221 is illustrated as a TIJ nozzle with an opening for ejection, examples are not so limited. Pump 221 may similarly be a PIJ resistor or other type of embedded pump.

FIG. 2B illustrates an example apparatus 200 for identifying target nucleic acids using immobilized nuclease, consistent with the present disclosure. Particularly, FIG. 2B illustrates the apparatus 200 including a plurality of heating elements. In examples in which nucleic acid amplification is to be used, the microfluidic channel 211 includes a thermo-cycling region 223 for nucleic acid amplification, and the reagent includes polymerase and dNTPs. The thermo-cycling region 223 may include a heating element or a plurality of heating elements disposed within the microfluidic channel 211. A non-limiting example of a heating element includes a resistor.

As illustrated in FIG. 2B, each of the plurality of side channels 213 may include an immobilized nuclease. For instance, side channel 213-1 may include immobilized nuclease 214-1, side channel 213-2 may include immobilized nuclease 214-2, and side channel 213-3 may include immobilized nuclease 214-3. Each respective nuclease may be different, and/or the nucleases may be the same. As an illustration, each side channel includes a different respective nuclease immobilized therein, and each respective side channel includes a respective heater disposed proximal to the immobilized nuclease. Sample and reagent, including gRNA, may be drawn into the appropriate side channel by firing the associated nozzle.

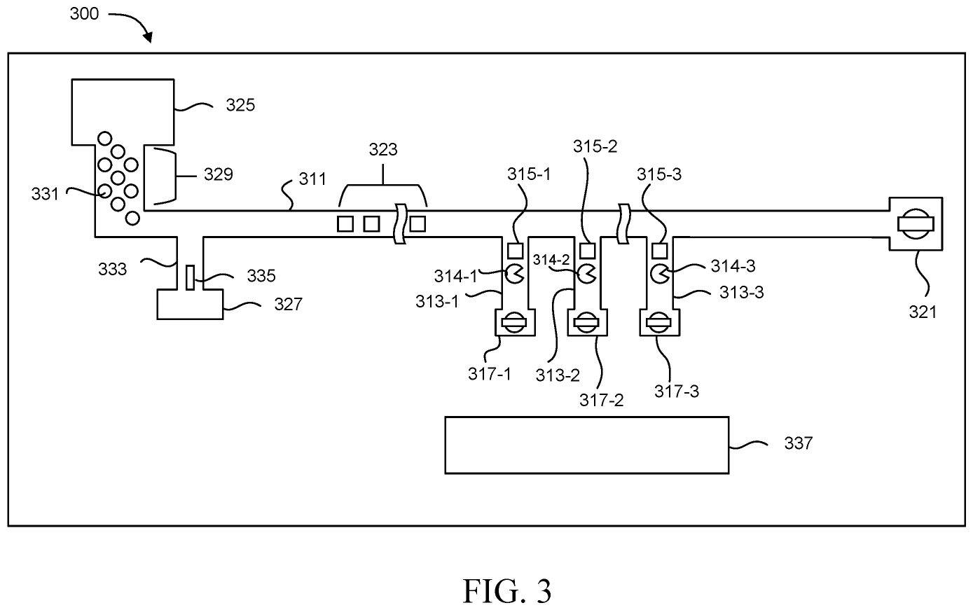

FIG. 3 illustrates an example apparatus 300 for identifying target nucleic acids using immobilized nuclease, including a sample reservoir and a reagent reservoir, consistent with the present disclosure. Similar components from FIG. 2A and FIG. 2B are used in FIG. 3 and numbered accordingly. For instance, a microfluidic channel 311 is fluidically coupled to a plurality of side channels 313-1, 313-2, and 313-3. Each of the plurality of side channels includes a respective pump 317-1, 317-2, and 317-3. Also, each of the plurality of side channels includes an immobilized nuclease 314-1, 314-2, and 314-3, as well as a heater 315-1, 315-2, and 315-3. As discussed with regards to FIG. 2B, a thermo-cycling region 323 including a plurality of heating elements is disposed within the microfluidic channel 311. The thermo-cycling region 323 may include more heating elements than illustrated, as indicated by the wave symbol within thermos-cycling region 323. Similarly, the apparatus 300 may include more side channels than illustrated, as indicated by the wave symbol disposed between side channel 313-2 and side channel 313-3. In yet further examples, the apparatus 300 includes fewer heating elements in thermo-cycling region 323 than illustrated, and/or fewer side channels than illustrated.

As illustrated in FIG. 3, the apparatus 300 may include a sample reservoir 325, and a reagent reservoir 327, among other components. As used herein, the sample reservoir 325 refers to or includes a chamber to store a volume of fluid sample to be tested. As used herein, the reagent reservoir 327 refers to or includes a chamber to store a volume of reagent or reagents. As discussed with regards to FIG. 2A and FIG. 2B, the microfluidic channel 311 may be coupled to a plurality of side channels 313. Each of the side channels 313 contains a nuclease and a guide RNA to program the nuclease. Each of the side channels 313 may include an integrated pump 317, such as a TIJ nozzle or push (inertial) pump to draw sample fluid from the sample reservoir 325, and reagents from the reagent reservoir 327. As illustrated, pump 335 may draw reagent from the reagent reservoir 327 for use in the microfluidic channel 311.

Upstream of the microfluidic channel 311 may be also a sample preparation chamber 329. The sample preparation chamber 329 may include a number of components, such as posts 331 or post-like structures for particle filtration. Particularly, the apparatus 300 includes a sample preparation chamber fluidically coupled to the microfluidic channel, wherein the sample preparation chamber includes a plurality of filtration structures disposed orthogonal to the flow of the fluid sample in the microfluidic channel. In some examples, the plurality of filtration structures include posts with affinity molecules disposed thereon.

The sample preparation chamber 329 may include posts with antibodies or other affinity molecules for removal of nuclease inhibitors. PCR and other nucleic acid amplification polymerases are sensitive to by-products or contaminants coming from a sample to be analyzed. Extraction and purification processes in the sample preparation chamber 329, may assist in reducing such contaminants prior to nucleic acid amplification, if used.

An example use of apparatus 300, including a sample preparation process, follows. During operation, integrated pull pumps (e.g., pumps 317) of each side channel 313 can fire sequentially or simultaneously to pull both the fluid sample and the reagents through a sample preparation region from the sample reservoir 325 and reagent reservoir 327, respectively. The sample preparation chamber 329 eliminates nuclease inhibitors and particles that may clog the channels (e.g., 311 and 313), and eliminates polymerase or other amplification inhibitors prior to amplification. A pump 335 draws reagent from the reagent reservoir 327. The sample from the sample reservoir 325 mixes with the reagents from the reagent reservoir 327, and in the example illustrated in FIG. 3, the sample undergoes amplification of the nucleic acid in thermo-cycling region 323. Although FIG. 3 illustrates a thermo-cycling region 323, example methods consistent with the present disclosure may be performed without nucleic acid amplification and therefore without use of the thermo-cycling region 323. The thermo-cycling region 323 may cycle through temperatures appropriate for a specified nucleic acid amplification protocol. The sample with the (optionally) amplified nucleic acid then travels to the immobilized nucleases in the side channels 313.

As discussed herein, the reagent may include a reporter nucleic acid labeled with a detectable ligand. As used herein, a detectable ligand refers to or includes a molecule that can be cleaved or otherwise deactivated by a nuclease described herein. Cleavage of the detectable ligand releases agents or produces conformational changes that allow a detectable signal to be produced. Prior to cleavage, the detectable ligand blocks the generation or detection of a positive detectable signal. In certain examples a minimal background signal may be produced in the presence of a reporter nucleic acid labeled with a detectable ligand. A positive detectable signal may be any signal that can be detected using optical, fluorescent, chemiluminescent, electrochemical or other detection methods. The term “positive detectable signal” is used to differentiate from background signals.

For example, as discussed with regards to example method 100 illustrated in FIG. 1, the method includes detecting the detectable ligand from the cleaved reporter nucleic acid in the side channel. In various examples, detecting the detectable ligand from the cleaved reporter nucleic acid in the side channel includes measuring a first signal in the side channel prior to receiving the fluid sample in the microfluidic channel, and measuring a second signal in the side channel responsive to receiving the fluid sample in the microfluidic channel. The first signal may correspond with a background signal, and the second signal may correspond with a test signal. The method further includes identifying the target nucleic acid in the fluid sample responsive to the second signal exceeding the first signal. Additionally and/or alternatively, the target nucleic acid may be identified in the fluid sample responsive to the second signal exceeding the first signal. The signal threshold includes a level of fluorescence or a change in current, as compared to the background signal, that meets or exceeds a value associated with a positive detectable signal.

An example of a detectable ligand that may be used to label a reporter nucleic acid includes a fluorophore and a quencher of the fluorophore. Quenching of the fluorophore can occur as a result of the formation of a non-fluorescent complex between the fluorophore and another fluorophore or non-fluorescent molecule. This mechanism is referred to as ground-state complex formation, static quenching, or contact quenching. Accordingly, this detectable ligand may be designed so that the fluorophore and quencher are in sufficient proximity to the immobilized nuclease for contact quenching to occur. Upon activation of the nuclease as disclosed herein, the detectable ligand is cleaved thereby severing the proximity between the fluorophore and quencher.

Upon contact with the target, the nucleases begin to indiscriminately cut nucleic acids (e.g., collateral cleavage), specifically short quencher-fluorophore pairs nearby the binding and cis-cleavage site of the nuclease. As used herein, collateral cleavage refers to or includes non-specific cleavage of nearby single-stranded nucleic acids by the activated programmable nuclease, such as collateral cleavage of detectable ligands. Once the detectable ligand is cleaved by the activated programmable nuclease, a signal may be generated. A signal can be a calorimetric, potentiometric, amperometric, optical (e.g., fluorescent, colorometric, etc.), or piezo-electric signal. This cleavage activity is triggered along with the binding and cleavage of the DNA duplexes by the nuclease and gRNA complexes, thereby leading to the generation of appreciable fluorescence signals in the presence of the target nucleic acid sequence associated with the particular nuclease. When uncut, the quencher blocks fluorescence and no florescence is observed by fluorimeter 337. When cut, the quencher and the fluorophore pair are separated and fluorescence is observed by the fluorimeter, indicating presence of the target.

The fluorescence may be read in the device or may be ejected by the nozzle for an off chip fluorimeter detection. For instance, in various examples, the fluid sample may remain in the respective side channel and fluorescent signals may be read in the side channel before dispensing. As an illustration, fluid sample may remain in any one (or multiple of) side channel 313-1, side channel 313-2, and 313-3, and the fluorimeter 337 may read the fluorescence signal from each prior to ejection out of nozzles 317-1, 317-2, and 317-3. Additionally and/or alternatively, the fluorescence may be read off of apparatus 300, such as by dispensing the fluid sample into a microwell and measuring the fluorescence in the respective microwell. As an illustration, nozzle 317-1 may eject a fluid sample into a first microwell, nozzle 317-2 may eject a fluid sample into a second microwell, and nozzle 317-3 may eject a fluid sample into a third microwell. After dispensing the fluid sample into the respective microwell, an off chip fluorimeter (e.g., a fluorimeter other than 337 illustrated in FIG. 3) may detect the level of fluorescence from each sample and therefore detect the presence of target nucleic acid in each respective microwell. As discussed with regards to FIG. 2A and FIG. 2B, nozzle 321 may eject any excess or additional fluid sample.

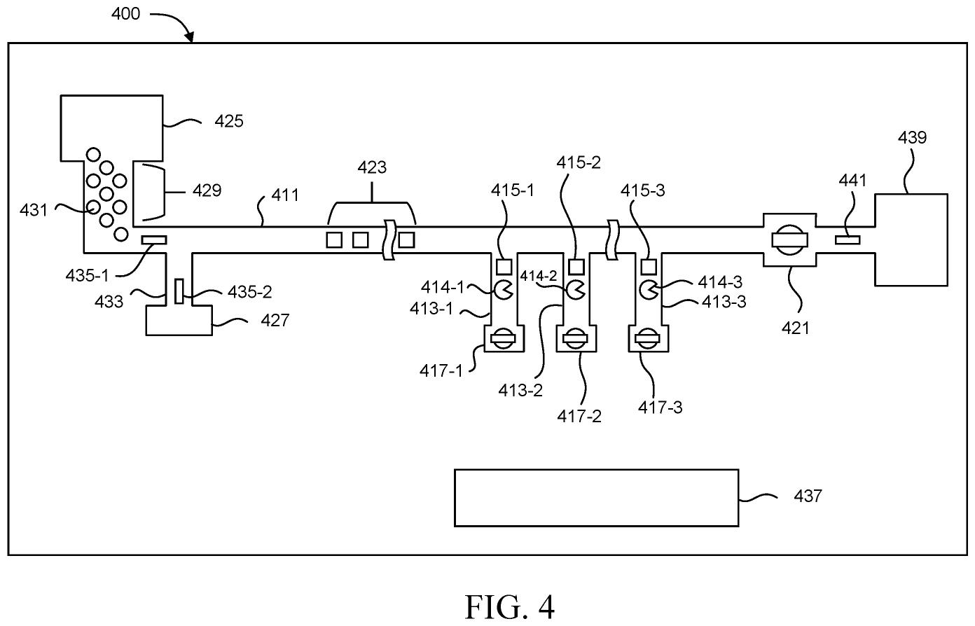

FIG. 4 illustrates an example apparatus 400 for identifying target nucleic acids using immobilized nuclease, including a sample reservoir and a reagent reservoir, and a collector, consistent with the present disclosure. The apparatus 400 is capable of performing the method 100 illustrated in FIG. 1. Similar to the example illustrated in FIG. 3, the apparatus 400 includes a microfluidic channel 411 that is fluidically coupled to a plurality of side channels 413-1, 413-2, and 413-3. Each of the plurality of side channels includes a respective pump 417-1, 417-2, and 417-3. Also, each of the plurality of side channels includes an immobilized nuclease 414-1, 414-2, and 414-3, as well as a heater 415-1, 415-2, and 415-3, and a thermo-cycling region 423 including a plurality of heating elements is disposed within the microfluidic channel 411. As described with regards to FIG. 3, the apparatus 400 includes a sample reservoir 425, a reagent reservoir 427, a sample preparation region 429 including a plurality of posts 431, and a fluorimeter 437 to capture fluorescent signals during testing.

As illustrated, the apparatus 400 may further include a collector 439 to collect fluid sample for further (e.g., subsequent) testing. During operation, the fluid sample that is not ejected out of nozzles (e.g., pumps) 417-1, 417-2, or 417-3, may pass through microfluidic channel 411 to nozzle 421 and/or pump 441. Nozzle 421 may fire to eject the fluid sample therefrom, and pump 441 may fire (without firing of nozzle 421) to move the fluid sample into collector 439. The sample may then be ejected through the TIJ nozzle 441 and/or be deposited into collector 439. Additionally, pump 435-1 may be disposed within the microfluidic channel 411 and may move fluid sample through the microfluidic channel 411. Similarly, pump 435-2 may be disposed within channel 433 and may move reagent into the microfluidic channel. As such, pump 435-1 may pull sample from sample reservoir 425 and pump 435-2 may pull reagent from reagent reservoir 427.

In various examples, each side channel 313 includes a different respective nuclease 414 immobilized therein, and each respective side channel 313 includes a respective heater 415 disposed proximal to the immobilized nuclease. The nuclease in each side channel 313 may be immobilized in a location sufficiently close to the respective heater 415 such that the nuclease 414 and nucleic acid sample may be heated by the respective heater 415.

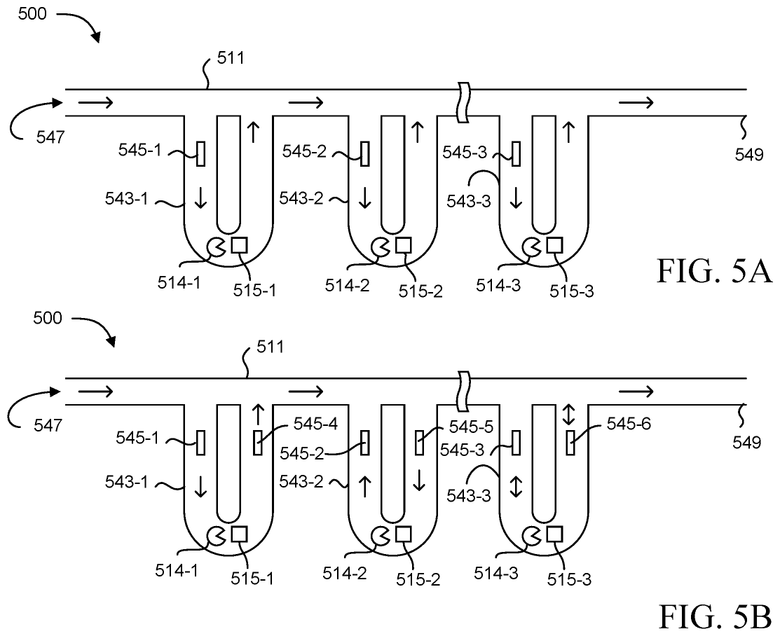

FIG. 5A illustrates an example apparatus 500 for identifying target nucleic acids, including a looped side channels, consistent with the present disclosure. The apparatus 500 is capable of performing the method 100 illustrated in FIG. 1. As illustrated in FIG. 5A, the apparatus 500 includes a microfluidic channel 511 including a sample input 547 to receive a fluid sample and a reagent input (either 547 or wax removal ports in each side channel) to receive a reagent for nucleic acid testing. In various examples, the reagent includes a reporter nucleic acid labeled with a detectable ligand. The apparatus 500 further includes a plurality of side channels, each side channel forming a loop fluidically coupled to the microfluidic channel. For instance, apparatus 500 includes side channels 543-1, 543-2, 543-3 (collectively referred to as side channels 543), each side channel forming a loop fluidically coupled to the microfluidic chamber 511. Each loop includes a pump, and a programmable nuclease immobilized in the respective side channel. For instance, side channel 543-1 includes pump 545-1 and nuclease 514-1, side channel 543-2 includes pump 545-2 and nuclease 514-2, and side channel 543-3 includes pump 545-3 and nuclease 514-3. As discussed with regards to FIG. 4, a heater may be disposed proximal to each respective nuclease. As an illustration, heater 515-1 may be disposed proximal to nuclease 514-1, heater 515-2 may be disposed proximal to nuclease 514-2, and heater 515-3 may be disposed proximal to nuclease 514-3. In various examples discussed herein, each side channel among the plurality of side channels includes a different respective programmable nuclease immobilized therein.

As illustrated, each side channel 543 forms a loop, and one side of the loop may include a pump to draw fluid into the respective side channel. For instance, a first side channel 543-1 includes with a first end fluidically coupled to the microfluidic channel 511, the first end including a pump 545-1. The pump 545-1 may draw the fluid from microfluidic channel 511 into the first side channel 543-1 and to the first nuclease 514-1. The sample may then flow in the direction of the arrow back to the microfluidic channel 511 to a second side channel 543-2. The second side channel 543-2 also includes a first end fluidically coupled to the microfluidic channel 511, the first end including a pump 545-2. The pump 545-2 may draw the fluid from microfluidic channel 511 into the second side channel 543-2 and to the second nuclease 514-2. The sample may then flow in the direction of the arrow back to the microfluidic channel 511 to a third side channel 543-3. The third side channel 543-3 also includes a first end fluidically coupled to the microfluidic channel 511, the first end including a pump 545-3. The pump 545-3 may draw the fluid from microfluidic channel 511 into the third side channel 543-3 and to the third nuclease 514-3. The fluid may flow in the direction of the arrow back to microfluidic channel 511 and out fluid output 549. In such a manner, a same fluid sample may be tested by a plurality of different nucleases in series, thereby reducing the amount of fluid sample needed and increasing the number of assays that are performed.

FIG. 5B illustrates an example apparatus 500 for identifying target nucleic acids, including a looped side channels, consistent with the present disclosure. Each side channel includes a first end fluidically coupled to the microfluidic channel, the first end including a first pump, and a second end fluidically coupled to the microfluidic channel at a different location than the first end, the second end including a second pump. For instance, side channel 543-1 includes pump 545-1 on the first side and pump 545-4 on the second side. Side channel 543-2 includes pump 545-2 on the first side and pump 545-5 on the second side. Side channel 543-3 includes pump 545-3 on the first side and pump 545-6 on the second side. The inclusion of pumps on opposing sides of each looped side channel 543 allows for agitation of the fluid sample. Agitation produces a reciprocal fluid flow, as indicated by the arrows, which increases efficiency and speed of the reaction.

Also, as discussed with regards to FIG. 5A, each side channel 543 includes a heater disposed at a distal end of the loop relative to the microfluidic channel 511. In such examples, the nuclease is disposed proximal to the heater. For instance, nuclease 514-1 is located at a distal end of side channel 543-1 relative to microfluidic channel 511, and heater 515-1 is located proximal to nuclease 514-1. Similarly, nuclease 514-2 is located at a distal end of side channel 543-2 relative to microfluidic channel 511, and heater 515-2 is located proximal to nuclease 514-2. Also, nuclease 514-3 is located at a distal end of side channel 543-3 relative to microfluidic channel 511, and heater 515-3 is located proximal to nuclease 514-3.

In the example illustrated in FIG. 5A and FIG. 5B, fluid sample enters the circulation loop, aided by the resistor (e.g., pump) in the circulation loop. The fluid sample is probed by the nuclease in the circulation loop. If the target nucleic acid is detected, the reporter DNA is cleaved by the nuclease and remains in the loop. If the target nucleic acid is not detected, the fluid sample is pumped out of the loop and into the microfluidic channel 511, to be pumped into the next loop. This allows the fluid sample to be probed for many sequences, without having to dilute the target by a large amount and therefore reducing the limit of detection performance. This is especially applicable in highly multiplexed assays including thousands of probing reactions, one nuclease of which is to match the fluid sample.

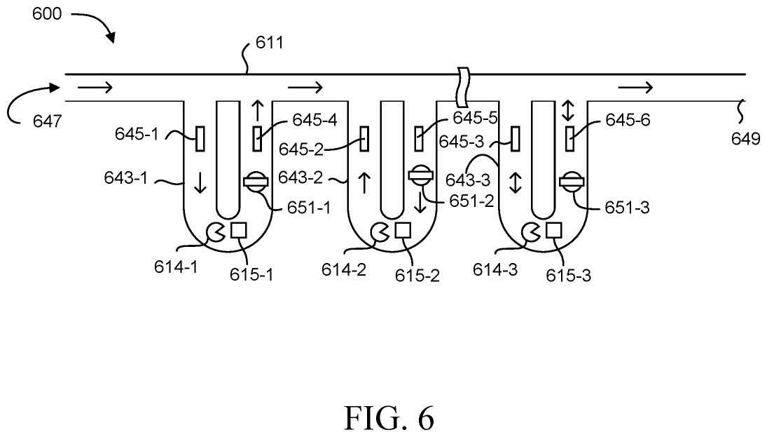

FIG. 6 illustrates an example apparatus 600 for identifying target nucleic acids, including looped side channels and additional pumps, consistent with the present disclosure. Apparatus 600 is capable of performing the method 100 illustrated in FIG. 1. Many of the same components illustrated in FIG. 5A and FIG. 5B are included in apparatus 600. For instance, apparatus 600 includes a fluid input 647, a fluid output 649, and a microfluidic channel 611. Apparatus 600 also includes a plurality of side channels 643-1, 643-2, 643-3, each forming a loop. Similar to FIG. 5B, each side channel includes a plurality of pumps. As an example, side channel 643-1 includes pump 645-1 and pump 645-4. Side channel 643-2 includes pump 645-2 and pump 645-5. Side channel 643-3 includes pump 645-3 and pump 645-6. Additionally, each side channel 643 includes a nuclease and a heater. As such, side channel 643-1 includes nuclease 614-1 and heater 615-1, side channel 643-2 includes nuclease 614-2 and heater 615-2, and side channel 643-3 includes nuclease 614-3 and heater 615-3. As indicated by the wave symbol between side channel 643-2 and 643-3, more side channels may be included than illustrated.

As illustrated in FIG. 6, each of the side channels may include TIJ nozzles to eject the sample out of the looped channel. Side channel 643-1 includes nozzle 651-1, side channel 643-2 includes nozzle 651-2, and side channel 643-3 includes nozzle 651-3. By including nozzles in each respective side channel 643, the fluid sample may either pass through to the next side channel in the sequence, or be ejected by the nozzle. For instance, if a target nucleic acid is detected by nuclease 614-1, the sample may be ejected out of nozzle 651-1 for collection, waste, and/or further testing. If the nucleic acid is not detected by nuclease 614-1, the sample may proceed to side channel 643-2, and so forth.

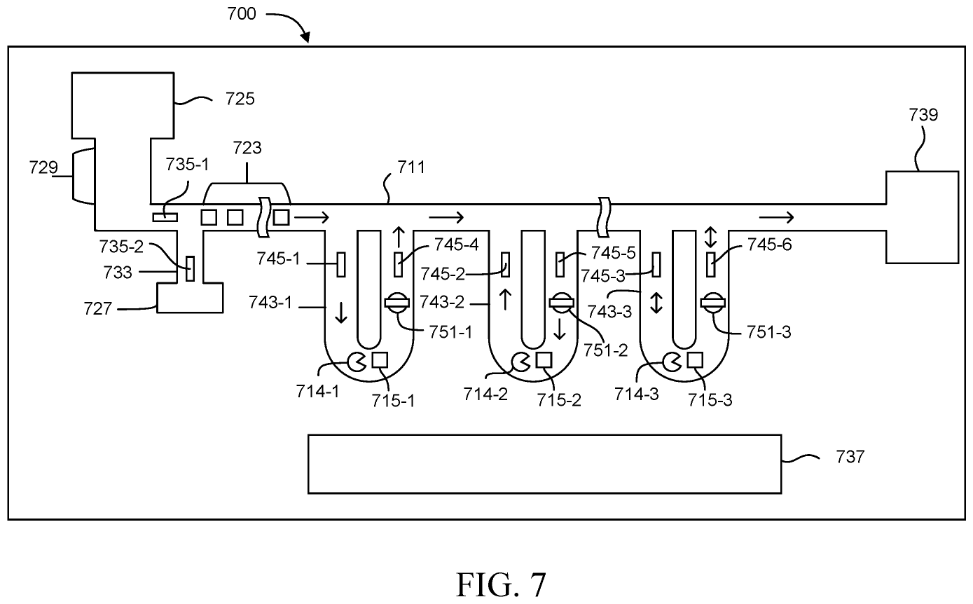

FIG. 7 illustrates an example apparatus 700 for identifying target nucleic acids, including looped side channels and additional pumps, consistent with the present disclosure. FIG. 7 illustrates the apparatus 700 including a sample reservoir 725, a reagent reservoir 727, and collector 739, coupled to the microfluidic channel 711. Similar to apparatus 400 illustrated in FIG. 4, the microfluidic channel 711 may include a thermo-cycling region 723, and a pump 735-1 to draw a sample out of sample reservoir 725. A sample preparation region 729 may (or may not) include a plurality of filtration structures. A pump 735-2 may be disposed within channel 733, and may draw reagent from reagent reservoir 727. A first side channel 743-1 includes a first pump 745-1 and a second pump 745-4, a nozzle 751-1, a nuclease 714-1 and a heater 715-1. The second side channel 743-2 includes a first pump 745-2 and a second pump 745-5, a nozzle 751-2, a nuclease 714-2 and a heater 715-2. The third side channel 743-3 includes a first pump 745-3 and a second pump 745-6, a nozzle 751-3, a nuclease 714-3 and a heater 715-4. A fluorimeter 737 is coupled to the apparatus and allows a sample to be read from the microfluidic channel.

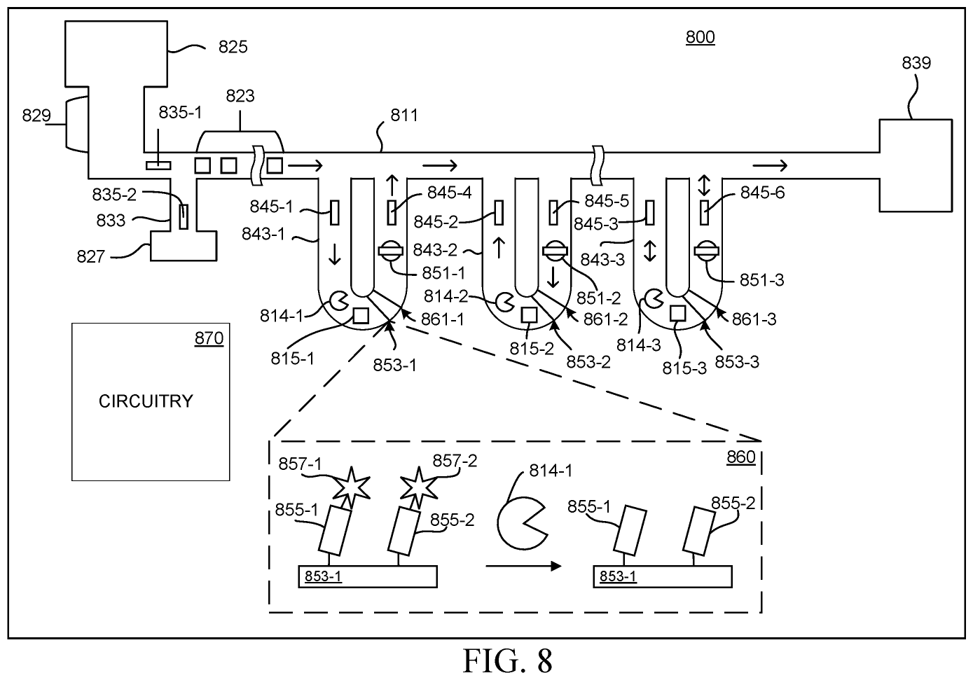

FIG. 8 illustrates an example apparatus 800 for identifying target nucleic acids, including looped side channels and electrochemical reporters, consistent with the present disclosure. While various figures have illustrated use of a fluorimeter, as well as fluorophore and a quencher, for detecting activity by a nuclease, examples are not so limited. For instance, as illustrated in FIG. 8, an electrochemical report molecule may be used to detect activity of a nuclease, and by extension, presence of a nucleic acid

Apparatus 800 includes most of the same components as apparatus 700, including a sample reservoir 825, a reagent reservoir 827, and collector 839, coupled to the microfluidic channel 811. The microfluidic channel 811 includes a thermo-cycling region 823, and a pump 835-1 to draw a sample out of sample reservoir 825. A sample preparation region 829 may (or may not) include a plurality of filtration structures. A pump 835-2 is disposed within channel 833, and may draw reagent from reagent reservoir 827. A first side channel 843-1 includes a first pump 845-1 and a second pump 845-4, a nozzle 851-1, a nuclease 814-1 and a heater 815-1. The second side channel 843-2 includes a first pump 845-2 and a second pump 845-5, a nozzle 851-2, a nuclease 814-2 and a heater 815-2. The third side channel 843-3 includes a first pump 845-3 and a second pump 845-6, a nozzle 851-3, a nuclease 814-3 and a heater 815-4.

As illustrated in the blow-up box 860, each side channel may include a plurality of electrodes for detecting the presence of a nucleic acid. For instance, the region near the nuclease may include a working electrode 853-1, 853-2, and 853-3, and a counter (not illustrated) as well as a reference electrode 861-1, 861-2, and 861-3 disposed nearby the working electrode 853-1, 853-2, and 853-3. Box 860 illustrates a blow-up view of working electrode 853-1. As illustrated in box 860, the working electrode 853-1 includes reporter DNA 855-1 and 855-2 coupled thereto, with an electrochemical reporter molecule 857-1 and 857-2 disposed at the distal end of the reporter DNA. Non-limiting examples of electrochemical reporter molecules include anthraquinone, Nile blue, and ferrocene. Once the nuclease (814-1 illustrated in box 860) is activated by presence of target DNA, it cleaves the reporter DNA molecules (as indicated in the reaction illustrated in box 860) releasing the electrochemical reporter molecule 857-1 and 857-2. Cleavage of the electrochemical reporter molecule drops the oxidation (reduction) current at the oxidation (reduction) potential of the electrochemical reporter respectively, indicating presence of target nucleic acid.

As such, in various examples, the apparatus 800 may include a working electrode disposed proximal to the nuclease, wherein the working electrode is functionalized with the reporter nucleic acid, and wherein the detectable ligand is an electrochemical reporter. The apparatus 800 may further include a counter, and a reference electrode, as well as circuitry 870 communicatively coupled to the working electrode, the counter, and the reference electrode to detect presence of a target nucleic acid via a drop in oxidation current.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A method, comprising:
receiving in a microfluidic channel, a fluid sample and a reagent, wherein the reagent includes a reporter nucleic acid labeled with a detectable ligand;
identifying a target nucleic acid in the fluid sample using a guide ribonucleic acid (gRNA) and a programmable nuclease immobilized in a side channel fluidically coupled to the microfluidic channel;
responsive to identifying the target nucleic acid in the fluid sample, causing cleavage of the detectable ligand; and
detecting the detectable ligand from the cleaved reporter nucleic acid in the side channel.

2. The method of claim 1, wherein a plurality of side channels are fluidically coupled to the microfluidic channel, each side channel including a respective pump, and wherein the method includes moving the fluid sample into each of the plurality of side channels by actuating the pumps in the side channels.

3. The method of claim 2, further including moving the fluid sample into each of the plurality of side channels in a sequential order by independently actuating the respective pumps in the sequential order.

4. The method of claim 1, including filtering, using plurality of filtration structures disposed orthogonal to the flow of the fluid sample in the microfluidic channel, polymerase or nuclease inhibiting compounds.

5. The method of claim 1, wherein the detectable ligand is a fluorophore, and the reporter nucleic acid is labeled with the fluorophore and a quencher, and wherein detecting the detectable ligand from the cleaved reporter nucleic acid in the side channel includes:
measuring a first signal in the side channel prior to receiving the fluid sample in the microfluidic channel; and
measuring a second signal in the side channel responsive to receiving the fluid sample in the microfluidic channel; and
identifying the target nucleic acid in the fluid sample responsive to the second signal exceeding the first signal.

6. The method of claim 1, wherein the detectable ligand is an electrochemical reporter probe.

7. An apparatus, comprising:
a sample input to receive a fluid sample;
a reagent input to receive a reagent for nucleic acid testing, wherein the reagent includes a reporter nucleic acid labeled with a detectable ligand;
a microfluidic channel fluidically coupled to the sample input and the reagent input; and
a plurality of side channels fluidically coupled to the microfluidic channel, wherein each side channel includes a guide ribonucleic acid (gRNA) and a programmable nuclease immobilized in the respective side channel.

8. The apparatus of claim 7, further including a sample preparation chamber fluidically coupled to the microfluidic channel, wherein the sample preparation chamber includes a plurality of filtration structures disposed orthogonal to the flow of the fluid sample in the microfluidic channel.

9. The apparatus of claim 8, wherein the plurality of filtration structures include posts with affinity molecules disposed thereon.

10. The apparatus of claim 7, wherein the microfluidic channel includes a thermo-cycling region for nucleic acid amplification, and wherein the reagent includes polymerase and deoxyribonucleotide triphosphates (dNTPs).

11. The apparatus of claim 7, wherein each side channel includes a different respective nuclease immobilized therein, and each respective side channel includes a respective heater disposed proximal to the immobilized nuclease.

12. An apparatus, comprising:
a microfluidic channel including a sample input to receive a fluid sample and a reagent input to receive a reagent for nucleic acid testing, wherein the reagent includes a reporter nucleic acid labeled with a detectable ligand;
a plurality of side channels, each side channel forming a loop fluidically coupled to the microfluidic channel, wherein each loop includes:
a pump; and
a programmable nuclease immobilized in the respective side channel.

13. The apparatus of claim 12, wherein each side channel among the plurality of side channels includes a different respective programmable nuclease immobilized therein.

14. The apparatus of claim 12, wherein each side channel includes:
a first end fluidically coupled to the microfluidic channel, the first end including a first pump;
a second end fluidically coupled to the microfluidic channel at a different location than the first end, the second end including a second pump; and
a heater disposed at a distal end of the loop relative to the microfluidic channel;
wherein the nuclease is disposed proximal to the heater.

15. The apparatus of claim 12, wherein each side channel includes:
a working electrode disposed proximal to the nuclease, wherein the working electrode is functionalized with the reporter nucleic acid, and wherein the detectable ligand is an electrochemical reporter;
a counter; and
a reference electrode;
wherein the apparatus further includes circuitry communicatively coupled to the working electrode, the counter, and the reference electrode to detect presence of a target nucleic acid via a drop in oxidation current.
